(19) United States
(12) Patent Application Publication
(10) Pub. No.: US 2025/0361199 A1
(43) Pub. Date: Nov. 27, 2025

(54) BIOGAS FEED FOR PRODUCTION OF ACETIC ACID

(71) Applicant: Topsoe A/S

(72) Inventors: Martin ØSTBERG, Tune (DK); Peter Mølgaard MORTENSEN, Roskilde (DK); Troels Juel FRIIS-CHRISTENSEN, Smørum (DK)

(73) Assignee: Topsoe A/S, Kgs. Lyngby (DK)

(21) Appl. No.: 18/874,684

(22) PCT Filed: Jun. 15, 2023
(86) PCT No.: PCT/EP2023/066149

§ 371 Date: Dec. 13, 2024

(30) Foreign Application Priority Data
Jun. 17, 2022 (EP) 22179666.7

(51) Int. Cl. C07C 51/15; C07C 51/44

(52) U.S. Cl. CPC C07C 51/15; C07C 51/44

(57) ABSTRACT

An acetic acid plant and associated process is provided, in which a biogas stream is fed to a reformer section, in which a first synthesis gas stream is provided. An acetic acid synthesis section is arranged to receive a synthesis gas stream from the reformer section and provide a raw acetic acid product stream.

### TECHNICAL FIELD

The present invention relates to a plant and process for production of acetic acid from biogas.

### BACKGROUND

Biogas is a renewable energy source that can be used for heating, electricity, and many other operations. Biogas can be cleaned and upgraded to natural gas standards, to become bio-methane. Biogas is considered to be a renewable resource because its production-and-use cycle is continuous, and it generates no net carbon dioxide. When the organic material has grown, it is converted and used. It then regrows in a continually repeating cycle. From a carbon perspective, as much carbon dioxide is absorbed from the atmosphere in the growth of the primary bio-resource as is released, when the material is ultimately converted to energy.

Biogas is a mixture of gases produced by the breakdown of organic matter in the absence of oxygen. Biogas can be produced from raw materials such as agricultural waste, manure, municipal waste, plant material, sewage, green waste or food waste. Biogas is primarily methane (CH₄) and carbon dioxide (CO₂) and may include small amounts of hydrogen sulfide (H₂S), moisture, siloxanes, and possibly other components.

A biogas contains typically about 50-60% methane and 40-50% CO₂. To utilize the CO₂ in the biogas, it is advantage to produce a syngas that can be fed to a downstream synthesis that takes advantage of the H₂/CO ratio that can be obtained.

A process and plant for converting biogas to methanol is described in WO2020254121. A method for converting syngas to acetic acid is provided in U.S. Pat. No. 4,584,322A.

The synthesis gas (syngas) produced from a biogas feed will contain a substantial amount of CO and remaining CO₂ as a consequence of the high CO₂ content in the biogas. This results in a synthesis gas with a low H₂/CO ratio lower than required for e.g. FT synthesis or MeOH synthesis. It would be desirable to provide chemical plants for effective use of biogas, which takes advantage of the H₂/CO ratio that biogas provides, preferably without having to adjust this ratio by e.g. adding extra hydrogen.

### SUMMARY

It has been discovered by the present inventor(s) that the H₂/CO ratio of a syngas obtainable from reforming of biogas is optimal for acetic acid synthesis.

So, in a first aspect the present invention relates to an acetic acid plant, said plant comprising:
a biogas stream,
a reformer section arranged to receive at least a portion of the biogas stream and provide a first synthesis gas stream,
an acetic acid synthesis section, arranged to receive a synthesis gas stream from the reformer section and provide a raw acetic acid product stream.

A process is also described for providing an acetic acid product stream from a biogas stream in an acetic acid plant, said process comprising:
providing at least a portion of the biogas stream to the reformer section to provide a first synthesis gas stream,
providing a synthesis gas stream from the reformer section to the acetic acid synthesis section, and providing a raw acetic acid product stream.

Further details of the technology are provided in the enclosed dependent claims, figures and examples.

### LEGENDS TO THE FIGURES

The technology is illustrated by means of the following schematic illustrations, in which:

FIG. 1 shows a schematic process layout of the plant of the invention.

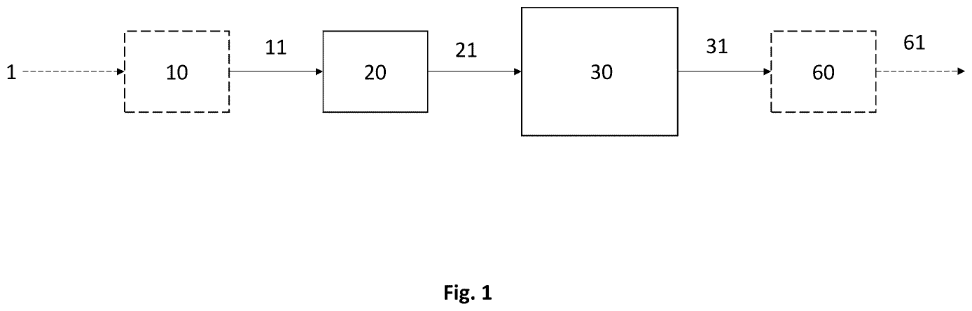

### DETAILED DISCLOSURE

Unless otherwise specified, any given percentages for gas content are % by volume. All feeds are preheated as required.

The term “synthesis gas” is meant to denote a gas comprising hydrogen, carbon monoxide and also carbon dioxide and small amounts of other gasses, such as argon, nitrogen, methane, etc.

As noted above, synthesis gas (syngas) produced from a biogas feed will contain a substantial amount of CO and remaining CO₂ as a consequence of the high CO₂ content in the biogas. This results in a synthesis gas with a lower H₂/CO ratio lower than that which is required for e.g. FT synthesis or MeOH synthesis. However, the present inventors have realised that—in the case where acetic acid is the desired product from the downstream synthesis—the module for this reaction Mac=(γ_{H2}−γ_{co2})/(γ_{co}+γ_{co2}) as is also known from MeOH synthesis should optimally be just above 1. This is because the overall reaction forming acetic acid is:

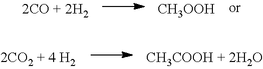

This can be obtained from a biogas feed performing a reforming (preferably an electrical reforming (eSMR) step on the biogas feed, with a low steam to carbon ratio. A biogas feed of 55% CH₄ and 45% CO₂ with addition of 2% hydrogen will provide a module of 1.25 (H₂/CO ratio of 1.54) at a steam to carbon ratio of 1.1 reformed to a temperature of 950° C. with a 30° C. approach. This is very low for both FT synthesis and MeOH synthesis, but close to optimal for an acetic acid synthesis, when it is produced directly from the synthesis gas as known in e.g. the BP SaBree process which is a technology for the production of acetic acid from syngas. This SaBree process converts synthesis gas (carbon monoxide and hydrogen derived from hydrocarbons such as natural gas) directly to acetic acid in an integrated three-step process that avoids the need to purify carbon monoxide (CO) or purchase methanol, according to BP. SaaBre process is expected to deliver a significant reduction in variable manufacturing costs, and lead to capital efficiencies, compared to the carbonylation of methanol route which has been the leading technology for several decades.

An acetic acid plant is thus provided, in which a biogas stream is converted to an acetic acid product stream. In general terms, the plant comprises:
a biogas stream,
a reformer section, and
an acetic acid synthesis section.

Also, the plant may further comprise:
a first biomass feed,
a biomass digester, arranged to receive the first biomass feed and convert it to a biogas stream,
wherein the reformer section is arranged to receive at least a portion of the biogas stream from the biomass digester.

These components, their arrangement and their function will be discussed in detail in the following.

### Biomass Feed

A biomass feed is typically a liquid slurry, with a total solids content of between 20-40%. Apart from water, biomass principally comprises organic material which can be converted by the action of microbes to a biogas, e.g. in an anaerobic digestion with anaerobic organisms or methanogen inside an anaerobic digester. Sources of biomass feed include agricultural waste, such as manure, sewage, green waste and food waste, as well as industrial waste e.g. from food or drink production.

Apparatus for handling and supply of the biomass feed to the plant are known to the skilled engineer.

### Biomass Digester

A biomass digester is arranged to receive the first biomass feed and provide a biogas stream. The term “biogas” in connection with the present invention denotes a gas with the following composition:

| Compound | % |
|---|---|
| Methane | 50-75 |
| Carbon dioxide | 25-50 |
| Nitrogen | 0-10 |
| Hydrogen | 0-1 |
| Oxygen | 0-1 |

The bacteria which convert the biomass feed into biogas are capable of digesting most hydrocarbon feedstocks. This is important in the combination of a biogas unit with a chemical synthesis unit.

A biomass digester is typically in the form of a pressure reaction vessel with appropriate inlet(s) for biomass and outlet(s) for biogas. Additional inlets and outlets may be provided for the various waste water streams recycled according to the invention. Inlets and outlets may also be provided for e.g. sampling the contents of the digester or introducing or removing microbial matter.

The biomass digester operates most effectively at around 50° C. In one aspect, the plant comprises means for heating the biomass digester, preferably a heat exchanger.

In one aspect, at least a portion of the first and/or at least a portion of the second off-gas stream, or a combination of the first and second off-gas streams, is arranged to be fed through said heat exchanger, thereby heating the biomass digester. This makes effective use of heat energy in the off-gas streams.

Additionally, the reformer section and/or the synthesis section may comprise one or more heat exchangers, arranged to exchange heat between one or more cooling streams in said plant and one or more streams in said reformer section and/or said synthesis section; and thus provide one or more heated streams from said cooling streams, and wherein at least a portion of said heated stream(s) is arranged to heat the biomass digester. In this manner, off-gas streams may be used to heat the reformer section and/or the synthesis section (which may have a high heat requirement) before they are sent (at a lower temperature) to the biomass digester.

Compared to a non-heated biomass digester, a heated biomass digester provides a lower residence time in the vessel, and therefore a high production.

Direct heating with steam has the disadvantage of requiring an elaborate steam-generating system (including desalination and ion exchange as water pre-treatment) and can also cause local overheating. The high cost may only be justifiable for large-scale sewage treatment facilities. The injection of hot water raises the water content of the slurry and should only be practiced if such dilution is necessary.

Indirect heating is accomplished with heat exchangers located either inside or outside of the digester, depending on the shape of the vessel, the type of substrate used, and the nature of the operating mode.
1. Floor heating systems have not served well in the past, because the accumulation of sediment gradually hampers the transfer of heat.
2. In-vessel heat exchangers are a good solution from the standpoint of heat transfer as long as they are able to withstand the mechanical stress caused by the mixer, circulating pump, etc. The larger the heat-exchange surface, the more uniformly heat distribution can be effected which is better for the biological process.
3. On-vessel heat exchangers with the heat conductors located in or on the vessel walls are inferior to in-vessel-exchangers as far as heat-transfer efficiency is concerned, since too much heat is lost to the surroundings. On the other hand, practically the entire wall area of the vessel can be used as a heat-transfer surface, and there are no obstructions in the vessel to impede the flow of slurry.
4. Ex-vessel heat exchangers offer the advantage of easy access for cleaning and maintenance.

Further components and design of the biomass digester are known to the skilled engineer.

### Reformer Section

A reformer section is arranged to receive at least a portion of the biogas stream and provide a first synthesis gas stream.

The first synthesis gas stream typically comprises (in % by volume)
0.5-5% methane (dry)
40-70% H₂ (dry)
10-30% CO (dry)
2-20% CO₂ (dry)

The reformer section may comprise one or more of an autothermal reforming (ATR) unit, a steam methane reforming (SMR) unit and an electrically heated steam methane reforming (e-SMR) unit, and is preferably an electrically heated steam methane reforming (e-SMR) unit. The reformer section suitably comprises or consists of an electrical steam methane reforming (eSMR) unit. Details of an e-SMR unit that is preferably used in the reformer section are found in WO2020254121.

Additional feeds (e.g. a steam feed or oxygen-rich feed) are supplied to the reformer section, as required, depending on the type of reforming to be carried out. For instance, SMR requires a steam feed, while ATR requires a steam feed and an oxygen-rich feed.

A first waste water stream is typically also provided by the reformer section.

### Acetic Acid Synthesis Section

An acetic acid synthesis section is arranged to receive a synthesis gas stream from the reformer section and provide a raw acetic acid product stream.

Suitable apparatus and processes for acetic acid synthesis from a syngas stream are provided in e.g. WO19003213 and EP2918327.

### Distillation Section

A distillation section may be arranged to receive at least a portion of the raw acetic acid stream and provide a purified acetic acid stream.

Various layouts of the distillation section are possible. Typically, the distillation section comprises one or more distillation columns arranged in series, through which the raw acetic acid is passed.

### Process

The present technology also provides a process for providing an acetic acid product stream from a biogas stream in an acetic acid plant as described herein, said process comprising:
providing at least a portion of the biogas stream to the reformer section to provide a first synthesis gas stream,
providing a synthesis gas stream from the reformer section to the acetic acid synthesis section, and providing a raw acetic acid product stream.

In the case where the plant further comprises a first biomass feed and a biomass digester, the process may further comprise the steps of:
providing the first biomass feed to the biomass digester and converting it to a biogas stream, and
providing at least a portion of the biogas stream from the biomass digester to the reformer section.

All details of the plant described above are equally relevant for the process of the invention, mutatis mutandis.

The present invention has been described with reference to a number of embodiments and figures. However, the skilled person is able to select and combine various embodiments within the scope of the invention, which is defined by the appended claims. All documents mentioned herein are incorporated by reference.

1. An acetic acid plant, said plant comprising:
a biogas stream,
a reformer section configured to receive at least a portion of the biogas stream and provide a first synthesis gas stream,
an acetic acid synthesis section, configured to receive a synthesis gas stream from the reformer section and provide a raw acetic acid product stream.

2. The plant according to claim 1, further comprising:
a first biomass feed, and
a biomass digester configured to receive the first biomass feed and convert the first biomass to a biogas stream,
wherein the reformer section is configured to receive at least a portion of the biogas stream from the biomass digester.

3. The plant according to claim 1, wherein the reformer section comprises or consists of an electrical steam methane reforming unit.

4. The plant according to claim 1, further comprising a distillation section configured to receive at least a portion of the raw acetic acid stream and provide a purified acetic acid stream.

5. A process for providing an acetic acid product stream from a biogas stream in an acetic acid plant according to claim 1, said process comprising:
providing at least a portion of the biogas stream to the reformer section to provide a first synthesis gas stream,
providing a synthesis gas stream from the reformer section to the acetic acid synthesis section, and providing a raw acetic acid product stream.

6. The process according to claim 5, wherein said plant further comprises a first biomass feed and a biomass digester, said process further comprising;
providing the first biomass feed to the biomass digester and converting it to a biogas stream, and
providing at least a portion of the biogas stream from the biomass digester to the reformer section.

7. The plant according to claim 1, wherein the acetic acid synthesis section is configured to provide the raw acetic acid product stream by a synthesis reaction of the synthesis gas stream, wherein the synthesis gas stream has a Mac value in a range of 1 to 1.25, wherein Mac=(γ_{H2}−γ_{co2})/(γ_{co}+γ_{co2}).

8. The process according to claim 5, wherein the raw acetic acid product stream is provided by a synthesis reaction of the synthesis gas stream, wherein the synthesis gas stream has a Mac value in a range of 1 to 1.25, wherein Mac=(γ_{H2}−γ_{co2})/(γ_{co}+γ_{co2}).
